(19) United States
(12) Patent Application Publication
(10) Pub. No.: US 2019/0343770 A1
(43) Pub. Date: Nov. 14, 2019

(54) Hard Capsule Shell Compositions for the Oral Contraceptive Formulations

(71) Applicants: Hemant N. Joshi, Parsippany, NJ (US); Amitkumar Lad, Dumont, NJ (US)

(72) Inventors: Hemant N. Joshi, Parsippany, NJ (US); Amitkumar Lad, Dumont, NJ (US)

(21) Appl. No.: 15/975,270

(22) Filed: May 9, 2018

(51) Int. Cl. A61K 9/48; A61K 31/567; A61K 47/02; A61K 47/22; A61K 47/40; A61P 15/18

(52) U.S. Cl. CPC A61K 9/4816; A61K 9/4808; A61K 31/567; A61P 15/18; A61K 47/22; A61K 47/40; A61K 47/02

(57) ABSTRACT

A hard shell capsule includes a body and a cap cooperatively defining a hollow core hard shell capsule. Each of the body and the cap has a composition that includes a polymer forming a hard polymer structure of the body and of the cap and comprises a drug. The body further comprises a therapeutically effective amount of drug A loaded throughout the composition; the cap further comprises a composition comprising a therapeutically effective amount of drug B loaded throughout the composition. The body and cap compositions together containing a therapeutically effective amount of the drugs A and B; said drugs being oral contraceptive agents. The core of the capsule is filled with therapeutically effective amount of a second drug(s), a dietary supplement, minerals, a complexing agent and other excipients. With the help of FIG. 2, the invention can be very well understood easily. Drugs A and B are selected from the group of oral contraceptives, but are not limited to, Cyproterone acetate, Estradiol, Oestradiol, Norethindrone acetate, Ethinyl Estradiol, Levonorgestrel, Dienogest, Drospirenone, Desogestrel, Ethynodiol, Diacetate, Mestranol, Nomegestrol acetate, Norgestrel, Norgestimate, Dienogest, Norelgestromin, Norethisterone, Gestodene, Oestradiol valerate, and Ethynodiol diacetate.

### RELATED APPLICATION

This patent application is a continuation in part of and claims the benefit of pending U.S. patent application Ser. No. 15/859,342 filed Dec. 30, 2017 titled “Physically Dispersed, Molecularly Dissolved and/or Chemically Bound Drug(s) in an Empty, Hard Capsule Shell Composition” which in turn is a continuation of and claims the benefit of U.S. patent application Ser. No. 14/280,677 titled “Physically Dispersed, Molecularly Dissolved and/or Chemically Bound Drug(s) in an Empty, Hard Capsule Shell Composition” filed May 19, 2014 and issued as U.S. Pat. No. 9,884,024 on Feb. 6, 2018 which in turn is a continuation-in-part of and claims the benefit of U.S. patent application Ser. No. 12/841,008 titled “Physically/Molecularly Distributed and/or Chemically Bound Medicaments In Empty, Hard Capsule Shells” filed Jul. 21, 2010 and issued as U.S. Pat. No. 8,728,521 on May 20, 2014, each of the above-identified priority applications and patents incorporated by reference as if fully set forth herein.

### FIELD OF THE DISCLOSURE

The disclosure relates to an improved or modified hard capsule shell, which is commonly used as a drug delivery system. This patent focuses on formulations of oral contraceptives.

### BACKGROUND OF THE DISCLOSURE

A drug is “a chemical substance used in the treatment, cure, prevention, or diagnosis of disease or used to otherwise enhance physical or mental well-being.

Medicament is a medicine, or a substance used in a therapy. In this patent application, the terms “drug” and “medicament” have been used interchangeably.

Oral contraceptives (birth control pills) are medications that prevent pregnancy. Contraceptives for women are currently available, but lot of research has been going on to develop a male contraceptive. This patent covers contraceptives for females and males. Using contraceptive pills is one of the methods of birth control. Oral contraceptives are hormonal preparations that may contain combinations of the hormones estrogen and progestin or progestin alone. Combinations of estrogen and progestin prevent pregnancy by inhibiting the release of the hormones luteinizing hormone (LH) and follicle stimulating hormone (FSH) from the pituitary gland in the brain. LH and FSH play key roles in the development of the egg and preparation of the lining of the uterus for implantation of the embryo. Progestin also makes the uterine mucus that surrounds the egg more difficult for sperm to penetrate and, therefore, for fertilization to take place. In some women, progestin inhibits ovulation (release of the egg).

There are different types of combination birth control pills that contain estrogen and progestin that are referred to as “monophasic,” “biphasic,” or “triphasic.” Monophasic birth control pills deliver the same amount of estrogen and progestin every day. Biphasic birth control pills deliver the same amount of estrogen every day for the first 21 days of the cycle. During the first half of the cycle, the progestin/estrogen ratio is lower to allow the lining of the uterus (endometrium) to thicken as it normally does during the menstrual cycle. During the second half of the cycle, the progestin/estrogen ratio is higher to allow the normal shedding of the lining of the uterus to occur. Triphasic birth control pills have constant or changing estrogen concentrations and varying progestin concentrations throughout the cycle. There is no evidence that bi- or triphasic oral contraceptives are safer or superior to monophasic oral contraceptives, or vice versa, in their effectiveness for the prevention of pregnancy. (reference: https://www.medicinenet.com/oral_contraceptives_birth_control_pills/article.htm#what_are_birth_control_pills_and_how_do_they_work_mechanism_of_action).

The following is the real-life example for the desogestrel/ethinyl estradiol tablet regimen:

TABLE 1
Doses for contraception for desogestrel and ethinyl estradiol

| Phase | Doses, once a day |
|---|---|
| Monophasic | Days 1 to 21: 0.15 mg desogestrel/0.03 mg ethinyl |
| | estradiol tablet |
| | Days 22 to 28: 1 inert tablet |
| Biphasic | Days 1 to 21: 0.15 mg desogestrel/0.02 mg ethinyl |
| | estradiol tablet |
| | Days 22 to 23: 1 inert tablet |
| | Days 24 to 28: 0.01 mg ethinyl estradiol tablet |
| Triphasic | Days 1 to 7: 0.1 mg desogestrel/0.025 mg ethinyl |
| | estradiol tablet |
| | Days 8 to 14: 0.125 mg desogestrel/0.025 mg ethinyl |
| | estradiol tablet |
| | Days 15 to 21: 0.15 mg desogestrel/0.025 mg ethinyl |
| | estradiol tablet |
| | Days 22 to 28: 1 inert tablet |

It is clear that based on the regimen, the patient has to take medicine with different strengths during the one-month treatment period.

A combination drug therapy has been gaining a lot of importance in recent times. The combination therapy in medicine involves administration of two or more therapeutically active ingredients. The active ingredient may be administered in a single dosage form or each active ingredient may be administered separately. The reasons of a combination therapy could be multiple—synergistic effects of drugs, reduction of side effect of the primary drug by an adjunct drug, avoidance of taking multiple tablets/capsules per day thereby savings on co-payment for different medicines and assurance of patient compliance to drug therapies.

In some embodiments of the combination drug therapy, the active ingredients are administered in a single dosage form. In those instances, it is important to show that different drugs combined in the same dosage form are stable during storage of the dosage form and should not interact physically or chemically with other drugs or excipients to produce degradation products. Also, each drug should show the desired release rate from the dosage form to get absorbed in sufficient quantities upon oral administration or release the drug to surrounding environment in case of other delivery routes.

Pharmaceutical capsule dosage forms are widely used in delivering drugs. Composition is the combination of parts or elements and it is the way in which something is put together. The composition of the capsule shell consists of a list of ingredients incorporated and their respective quantities.

There are main two types of capsules—hard shell capsules and soft-shell capsules.

Hard shell capsules, also called hard capsules herein, have two parts, the body and the cap. The body and the cap telescopically engage one another to define an interior volume of the hard capsule that typically holds dry ingredients, often in powder or granulated form. The body and the cap of a hard capsule have sufficient rigidity that the body and cap may form “snap connections” that resist separation of the body and cap after telescopic engagement.

Some types of hard shell capsules are formed from compositions that include polymers that have gelled or otherwise hardened and thereby form a polymeric system forming a hard polymer structure defining the shape of the body and cap. Polymers used to form polymeric systems that can form hard polymer structures include, but are not limited to, gelatin and carrageenan.

Soft-shell capsules, also called softgels herein, are one piece, hermetically sealed shells often made of a gelatin, a plasticizer, and water. The plasticizer softens the gelatin and makes it flexible and non-rigid. A soft-shell capsule typically holds an oil-based liquid, suspension, or semisolid that does not dissolve the capsule's gelatin.

The term “hard” for a substance or matter is defined as a solid, firm substance or matter which is resistant to pressure and with an endurance.

Polymer is defined as a large molecule, or macromolecule, composed of many repeated subunits. A system is defined as a set of connected things or parts forming a complex whole. A polymeric system means a system in which one of the major components (in terms of quantity and functionality) is a polymer.

A structure is the arrangement of and relations between the parts or elements of something complex. A structure has a form, shape, composition, formation or a constitution. A hard polymer structure is defined as a rigid structure with polymer as a major component.

Hard capsule polymeric structure indicates a structure with a shape of capsule which is solid and does not change with time, external pressure and it has a polymer or a mixture of polymers as the major component. A hard capsule has a body and cap. The cap is placed on the body and pressed to stay locked. In contrast, the soft polymer capsule structure is soft in nature and can change the shape by some pressure. Soft capsule shells have higher amounts of water/moisture compared to the hard-capsule shell.

The manufacturing process for the hard and soft capsule shells are different. As mentioned earlier, in the case of hard capsules, body and caps are prepared separately and are put together to form the entire capsule. Most of the soft capsules are prepared with gelatin. A solution of gelatin in water is prepared in a heated mixing tank. This also contains several excipients. In another tank, the filling material containing medicines is prepared. The filling material can be a solution (liquid or semi-solid) or a suspension. During the encapsulation stage, the fill material and gelatin shell ribbon come together to form the softgel capsule. The capsules are dried to remove excess moisture but retain desired amount of moisture. In contrast, hard shell capsule shells contain significantly lower percent moisture.

The term “essential” means absolutely necessary. The hard-shell capsule must have some essential physical and chemical properties. Property of a substance is a characteristic, which helps to identify, describe, define or quantify the substance. The capsule shell is thin. Empty capsules shells are packed in big plastic bags and should not break during transportation and storage. After filling drugs, the capsules are stored in small bottles or in the blister packs. Capsule shells should not break during transportation and storage in these primary packaging systems. For that, they should have sufficient elastic stiffness, tensile strength and should not be brittle. The drug incorporated in the shell matrix should not affect the essential physical and chemical properties. The shell matrix is expected to dissolve once reaching the stomach or if the capsule is enteric coated, should dissolve in the intestine. Some chemicals such as aldehydes are known to crosslink gelatin, reducing its dissolution rate. The drug should not affect the desired dissolution rate of the capsule shell. If the drug is hygroscopic, it may let capsule shell absorb more moisture than desired. Higher moisture level in the hard capsule can make them soft. In the worst-case scenario, capsules can become sticky and adhere to each other in the bottle.

The present disclosure relates to the hard-capsule shell composition. The capsules are normally prepared using gelatin and other excipients. In recent times, several polymers have been employed to manufacture hard shell capsules. In 1977, Christen and Cheng patented (U.S. Pat. No. 4,026,986) hard shell capsules manufactured using 2-hydroxyalkyl starch. It facilitated the production of shells and produced improved shells. WO 1997004755 (International application PCT/EP1996/003263) prepared hard gelatin capsules with internal or external polymer coating using the double dipping technique. The inventors used polyvinyl alcohol and polyvinyl acetate polymers along with necessary additives.

Gennadios invented non-gelatin capsules (U.S. Pat. No. 6,214,376) comprising k-carrageenan, water soluble plasticizer, and dextrins. The composition also included hydrolyzed starch as a variation. U.S. Pat. No. 6,517,865 claimed hard and soft capsules comprising of water soluble cellulose ethers, hydrocolloids and sequestering agents. The capsules also comprised of a coating with polymers including cellulose acetate phthalate, hypromellose phthalate etc.

In 2004 patent by Chen et al. (U.S. Pat. No. 6,752,953), authors described the usage of other polymers such as cellulose derivatives including cellulose, cellulose ester, methylcellulose, hydroxypropyl methylcellulose etc., acrylates including polyacrylate, polymethylacrylate, poly(methacrylate-methylmethacrylate) etc., and polyolefins including polyethylene, polypropylene, polyvinyl chloride, polyvinyl alcohol etc. to prepare capsules. The inventors described a heat-melting method to prepare the capsule shells.

U.S. Pat. No. 8,029,821 used low-substituted cellulose ether to produce hard capsules. The inventors also proposed a method for capsule preparation. The pins were dipped in the alkaline solution of low-substituted cellulose ether followed by dipping in an aqueous acid solution to form a gel (low-substituted cellulose ethers are soluble in alkaline medium and form a gel in the acidic environment). The pins covered with the gel were further washed with water before the drying step.

U.S. Pat. No. 6,949,256 used a mixture of kappa carrageenan and iota carrageenan. Kappa carrageenan is known to form a strong gel in the presence of potassium cations. However, these tend to be brittle and exhibit syneresis (exudating of liquid portion of the gel). Iota carrageenan reacted with calcium cations and formed a weaker and more flexible gel. In the U.S. Pat. No. 8,105,625, Rajewski and Haslam prepared hard shell capsules with pullulan, a plasticizer and a dissolution enhancing agent. The capsules were meant to dissolve in the mouth cavity (orally dissolving capsules).

US patent application #2008/0274187 prepared hard capsule compositions comprising carrageenan, locust bean gum, xanthan gum, sorbitol, and pullulan. These capsules eliminated the problem of cracking, embrittlement, chipping and deformation due to water loss and mechanical stress.

US patent application #2010/0168410 described a composition of hard capsules of hydroxypropyl methylcellulose and the process of dip-coating manufacture. The dipping pins were heated at 55-95° C. and the polymer solution was maintained at 1° C. to 10° C. below its gelling temperature.

McConville et al. (Eur. J. Pharmaceutics & Biopharm. 57: 541-549 (2004)) prepared a capsule filled with low-substituted hydroxypropyl cellulose in which a propanol tablet was placed followed by an erodible tablet containing hydroxypropyl methylcellulose (HPMC) and lactose manufactured by either direct and wet granulation technique. In this case, the body of the capsule was pre-coated with insoluble ethyl cellulose suggesting the drug release only after the dissolution of the cap of the capsule. In this case, the capsule shell was not loaded with the drug—propranolol.

In the US patent application #2004/0146559, a film was formed on the inner surface of the capsule shell. The shell may have different properties to alter the drug release rates. In this patent, the core and the shell were manufactured in situ. In the present disclosure, the empty hard capsule shell composition containing the drug may be produced by the manufacturers of empty capsules (such as, Capsugel, Universal capsules, and Shionogi capsules).

In the U.S. Pat. No. 6,709,427, microspheres were encapsulated to produce microcapsules. The core technology was to prepare microspheres. Microcapsule is a totally different kind of drug delivery technique compared to the hard-capsule shell dosage form. In the hard-capsule shell dosage form, the drug granules or powder or microspheres are filled in the capsule shell body on which the cap is fitted.

In the US patent application #2003/0104062, the capsules core was loaded with the drug. The “capsule core” is the empty space within the capsule shells and not the capsule shell composition. Hollow is defined as an empty space. A “hollow core hard shell capsule” means a hard shell capsule with an empty space in the core. The shell surrounding the drug-containing core governs the release rate (zero order) of the drug by diffusion mechanism due to its swelling. The shell also promoted gastric retention of the capsules by swelling upon the imbibition of gastric fluid to a size that was retained in the stomach during the fed mode.

In the US patent application #2003/0104062, no drug was incorporated in the capsule shell composition. In another embodiment, the drug was incorporated in the shell to produce a burst effect. In this case, the same drug is incorporated in the core and in the shell/casing. In this patent, the preparation of capsule shell was part of the manufacturing process in situ. The desired zero order release, the manufacturing process and drug combination are the key differences between US patent application #2003/0104062 and the current patent application.

U.S. Pat. No. 7,666,398 by Uhrich K described the composition and method of preparation of a polymeric drug delivery system using polyanhydrides. Polyanhydrides linked low molecular weight drugs containing a carboxylic acid group and an amine, thiol, alcohol or phenol group within their structure. The inventors did not disclose the capsule as the drug delivery system.

Yamamoto et al. in their U.S. Pat. No. 5,756,123 described a capsule shell comprising of HPMC, carrageenan as a gelling agent and calcium or potassium ions as the co-gelling agent. Carrageenan helped to improve the shapability of HPMC. Co-gelling agents assisted the gelation of carrageenan. Calcium ions were used for iota-carrageenan whereas potassium ions were used for kappa carrageenan. Carrageenan was not included in this composition as a therapeutically active agent. But it was used as part of the polymer structure of the capsule as a gelling agent to improve capsule properties.

Sakanishi et al in their US patent application #2006/0275361 described rapidly dissolving gelatin compositions. Although gelatin is water-soluble, it takes several minutes to dissolve in mouth to release their core material. The inventors added dissolution enhancing materials to the gelatin composition such as polyol plasticizer and water-insoluble particulate components. Polyols were selected from the group consisting of glycerin, propylene glycol, sorbitol, maltitol, xylitol, mannitol, erythritol, isomalt, lactitol and combinations thereof. One has to be very careful about understanding the definitions of “capsule shell-forming composition” and “core composition”. Gelatin, polyol and water-insoluble component formed a “capsule shell-forming composition” with which, the capsules were formed. The “core composition” was the one which was filled in the above-mentioned capsules. The “core composition” may include sweeteners, flavors, medicaments and other excipients. The patent quoted, “Core materials may include hydrophobic components, such as flavor oils or vegetable oils, as well as hydrophilic components”. Anyone knowledgeable in the preparation of making capsules will agree that oils are not preferred in the “capsule shell-forming composition”. The patent further quoted, “The core compositions, as previously mentioned may take a variety of physical forms, such as powders, granules, gels, pastes or liquids”. It was clear that the inventors intended to fill their rapidly dissolving gelatin capsules with the “core materials” prepared using the core composition, which included active medicaments. The inventors did not intend to add the “actives” in the “capsule shell-forming composition”. Also, the inventors did not teach to make capsules using polymeric materials such as HPMC (hydroxypropyl methylcellulose).

There are mainly two methods to prepare capsules: pin dip-coating and heat-melting. A liquid mass is produced by dissolving the capsule shell-forming compositions in a solvent system or by melting at an appropriate temperature. In the dip-method, plurality of pins maintained at a certain temperature is dipped in the solution and is withdrawn at a pre-determined rate while spinning. The pins coated with capsule composition are then dried at a gradual rate. The capsules (body and cap) are separated from the pins and are trimmed to an exact length. The method has been employed to prepare the body and cap of the capsules. The body and cap are joined or fitted together or cooperatively engaged and a logo is printed, if necessary.

InnerCap (http://www.innercap.com/) proposed combination capsules in which a capsule may contain another small capsule or a tablet along with granules (multiphase, multi-compartment capsule technology). The granules may be made up of beads or other forms, which may contain more than one type of drugs. This way, more than one type of drug may be combined in the same capsule. This is a different technology compared to the current disclosure.

Soft gelatin capsules are another form of capsules in which a liquid core material is filled. It has created a niche market such of its own in the drug delivery technology. In coming times, soft non-gelatin may be introduced in the market due to advent of new polymeric systems.

### BRIEF SUMMARY OF THE DISCLOSURE

The following are the key words used and the definition of various terms applicable to this disclosure.

The present disclosure proposes a design to incorporate drug(s) in the capsule shell-forming composition (body and cap). The term “drug(s)” is optionally plural. It means one or more drugs may be incorporated in the capsule shell-forming composition. Drugs in the compositions of cap and body of the capsules may be the same or may be different. Other medicaments in the form of granules, beads etc. can be filled in the capsules, which are termed as “core materials”. Thus, the same capsule may contain drugs in the core matrix and in the capsule shell composition.

There are two key phrases—“capsule shell-forming composition” and “capsule-core material/formulation”.

Capsule shell-forming composition described herein includes a composition suitable for forming empty hard shell capsules. Capsule shell-forming composition in this patent application comprises of gelatin or a suitable polymer or a mixture of polymers, and suitable excipients including plasticizer, coloring agent etc.

The term “comprising” or “comprises” is synonymous with “including”, “containing” and “characterized by”, is inclusive or open-ended. In some embodiments, drug/drugs may be added to the capsule shell-forming composition. The capsule shell-forming compositions may be different for capsule cap and capsule body.

An “excipient” as used herein is an inert substance added as diluents or vehicles or to give form or consistency or properties.

Excipients may also act as a preservative. A “preservative” is understood herein to mean certain embodiments which are substances added to inhibit chemical change or microbial growth. When the preservative inhibits microbial growth, such preservatives may include, but are not limited to sodium benzoate, methylparaben, propyl gallate BP, sorbic acid, chlorobutanol, dihydroacetic acid, monothioglycerol, potassium benzoate, propylparaben, benzoic acid, benzalkonium chloride, benzethonium chloride, benzyl alcohol, butylparaben, cetylpyridinium chloride, ethylenediamine, ethylparaben, thimerosal and potassium sorbate.

In certain embodiments, the capsule shell-forming composition may also contain an antioxidant. An “antioxidant” is understood herein to mean certain embodiments which are substances that inhibit oxidation. Such antioxidants include, but are not limited to, ascorbyl palmitate, butylated hydroxyanisole, butylated hydroxytoluene, potassium metabisulfite, and sodium metabisulfite.

In certain embodiments, the capsule shell-forming composition may also contain a flavoring agent. A “flavoring agent” is understood herein to mean certain embodiments which are substances that alter the flavor of the composition during oral consumption. A type of “flavoring agent” would be a sweetener. Preferred sweeteners can be natural or artificial. Flavoring agents can be chosen from a group commonly used in the pharmaceutical dosage forms.

In certain embodiments, the capsule shell-forming composition may also contain a buffering agent. A “buffering agent” is a weak acid or a weak base or a mixture of two, which helps to prevent fluctuations in the pH of the medium when exposed to an acidic or basic environment. Some of the examples of “buffering agents” may be, but not limited to, phosphate, citrate, borate and acetate.

A “capsule dosage form” defined here as a delivery system for a drug(s) prepared with suitable materials such as gelatin or polymers to form a capsule shell and has a shape as depicted in FIG. 2.

A “capsule shell” is referred to as a film-forming composition used to encapsulate an active moiety in a capsule dosage form. For this disclosure, the capsule shell-forming composition also contains a drug or drugs.

A “medicament” is an agent that promotes recovery from an ailment or an injury. Similar words to medicament are medicine, drugs, therapeutic agent and an active moiety.

The “therapeutically effective amount” is the amount of pharmaceutical or nutraceutical substance that treats, totally or partially, a disease state or alleviates one or more symptoms of the condition.

The “empty, hard shell capsule” as name suggests is hard, durable and smooth capsule. It retains its shape and it is dry in nature. As evident from the word “empty”, there is nothing inside core portion of the capsule shell when supplied by the capsule manufacturing company. Currently such “empty, hard shell capsule” compositions available in the market do not contain any drug(s). The hard-capsule shell comprises of two parts—a body in the core of which holds the contents of the dosage form (“capsule core formulation or capsule core material”), such as, powders/granules/beads/a tablet/a capsule and a cap, which fits on the body of the capsule shell and acts as a cover (U.S. Pat. Nos. 4,510,168 and 4,532,881).

The “capsule core material” may be prepared from a wide variety of materials including, but not limited to, sweeteners, flavors, medicaments, coloring agents, dispersing agents, lubricants and glidants etc. The “capsule core material” can be in the form of but not limited to powder, granules, beads, pellets, microspheres, micro-capsules, mini-tablets and mini-capsules. Someone may argue that the body is type of a core. However, people working in the field of pharmaceutical formulations will not confuse the “body” of the capsule shell with the material within the core of the capsule.

The “drug-loaded empty, hard shells capsules” means the empty, hard capsule shells in which a drug is loaded in the shell composition. In one embodiment of this patent, drug(s) loaded in the capsule shell forming composition is termed as “first” drug(s). Thus, the “first” drug(s) can be in the capsule shell forming composition for the cap or in the capsule shell forming composition for the body or in both. The “second” drug is not in the capsule shell forming composition, but it is in the capsule core material/formulation. The “first drug” can be one or more drugs. In Oral contraceptive formulations, normally two drugs are combined and here we are defining them as drug A and drug B. In another embodiment of the patent, one of the “first” drug(s) (drug A or B or A and B) is incorporated in the capsule shell forming composition of the cap and the other of the “first” drug (drug A or B) is incorporated in the capsule shell forming composition of the body. There core of the capsule shell may be empty or filled with the “second” drug or with a dietary supplement or with a complexing agent or other excipients or mixtures thereof.

The term “loaded” means the drug is either physically dispersed or molecularly dissolved in the shell composition or chemically bound to the polymeric material incorporated in the capsule shell-forming composition.

The term “physically dispersed” in the above sentence means, the drug is suspended as a powder, crystals or granules in the capsule shell-forming composition. In other words, drug(s) is in an insoluble form in the capsule-shell forming composition.

The term “molecularly dissolved” in the above sentence means, the drug(s) is dissolved molecularly in the capsule shell-forming composition. Undissolved particles less than 200 nm particle size are not visible to the naked eye and can be considered “dissolved”.

The term “chemically bound to the polymeric material” in the above sentence means, the drug is ion-paired or complexed or covalently bound to the gelatin or polymer used to form the capsule shell. These drug-loaded empty hard shell capsules are prepared by incorporating drug in the capsule shell-forming composition, which is then converted to a capsule shape (body and cap).

The key advantage of incorporation of drug(s) in the shell is to obtain a desired rate of release of the medicament, mainly for potent drugs. The rate of release will be controlled by the way in which the drug is embedded in the capsule shell-forming composition. Other advantage is to produce a combination drug delivery system. The concept can be used for the hard gelatin, hard non-gelatin, soft gelatin and soft non-gelatin capsules. The type of medicaments or drugs embedded in the capsule shell-forming composition can be from any therapeutic class but should be of low-dose or should be potent. The term “low-dose” is relative and in general, for the purpose of this disclosure, it may be less than 5 mg of dose. The drug must be stable in the capsule shell during manufacture and during appropriate storage conditions for the capsules.

The term “dietary supplement” is product taken orally that contains one or more ingredients such as vitamins, minerals that are intended to supplement one's diet and are not considered food.

The term “vitamin” is defined as organic compounds that are essential for normal growth and nutrition and are required in small quantities in the diet because they cannot be synthesized by the body.

The term “minerals” is describes as chemical element required as a nutrient by organisms to perform functions necessary for the life.

The term “complexing agent” is known as coordination compound. In chemistry, it is a compound in which independently existing molecules or ions of a nonmetal form coordinate bonds with a metal atom or ion. In another embodiments, complexing agents such as cyclodextrin (alpha-cyclodextrin, beta-cyclodextrin, gamma-cyclodextrin, sulfobutyl ether betacyclodextrin, hydroxypropyl betacyclodextrin, randomly methylated betacyclodextrin), and activated charcoal are filled in the capsules shells containing oral contraceptives. The complexing agent may modify the pharmacokinetic profile of the oral contraceptives.

Other objects and features of the disclosure will become apparent as the description proceeds, especially when taken in conjunction with the accompanying drawing sheets illustrating one or more illustrative embodiments.

This patent covers oral contraceptives. Currently, oral contraceptives are administered only to female but oral contraceptives for males are being developed. There are various oral contraceptive combination products available in the market. For the purpose of this patent, the following drug products are possible—1. Drug A in the cap of the capsule and drug B in the body of the capsule, 2. Drugs A and B are distributed in both cap and body of the capsule, 3. Drug A or B is distributed only in the cap of the capsule shell matrix and no drug in the body of the capsule, 4. Drug A or B is distributed only in the body of the capsule shell matrix and no drug in the cap of the capsule, and 5. Drug A or B is distributed in both cap and body of the capsule.

In another embodiment, drugs other than oral contraceptives or dietary supplements can be filled into the capsule shells containing oral contraceptive drugs in the capsule shell matrix.

### BRIEF DESCRIPTION OF THE DRAWINGS

FIG. 1 depicts a capsule cap, which cooperatively engages in the capsule body to form a hard shell capsule. FIG. 1 also shows the drug-loading in the capsule cap (“first” drug(s)). FIG. 1 shows loading of a single drug. But more than one drug can be loaded in the capsule shell too.

FIG. 2 depicts a hard shell capsule in which the capsule cap is cooperatively engaged in the capsule body. In this figure, both the capsule cap and body compositions are loaded with a drug. It also shows the “core material” of the capsule, which contains a second drug filled into the capsule.

### DETAILED DISCLOSURE

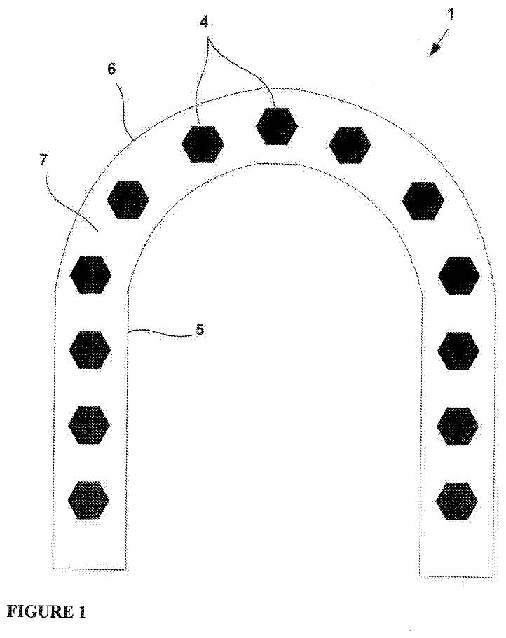

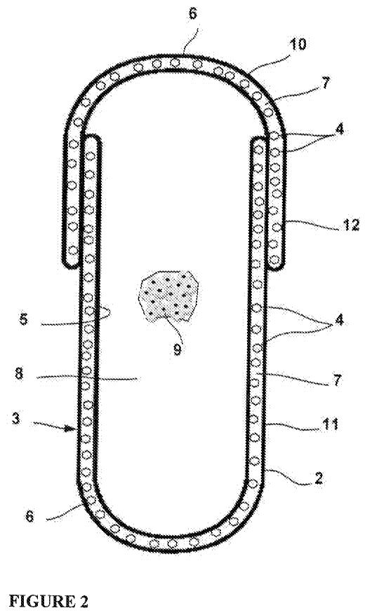

In accordance with the present disclosure, FIG. 1 depicts a Capsule Cap 1 that cooperatively engages a Capsule Body 2 to form a hard shell capsule 3 (see FIG. 2).

The Capsule Cap 1 in FIG. 1 has Medicament(s) or Drug Molecule(s) 4 dispersed throughout between a Capsule Cap inner surface 5 and a Capsule cap outer surface 6. The Capsule Body 2 in the illustrated embodiment of the hard shell Capsule 3 has a similar structure, that is, a drug dispersed throughout the Capsule Body 2 between inner and outer surfaces of the Capsule Body 2. The Shell Composition 7 of the Capsule Cap 1 or the Capsule Body 2 may consist of gelatin, HPMC, cellulose-derivatives, acrylates, polyolefins, vinyl polymers and other polymeric systems used in forming Caps and Body of conventional hard shell capsules. The main constituent of the composition 7 of the capsule shell (body or cap) may also be a combination of polymers mentioned above. The Shell Composition 7 (body or cap) may also contain other excipients such as plasticizers, emulsifiers, stabilizers, colorants, cross-linking agents etc. The rate of dissolution of capsule shell in a desired media or location within the body may be altered using appropriate capsule shell composition 7 as it is known in the art.

There are different ways of distribution of drug(s) in the capsule shell matrix. The Medicament or Drug 4 can be physically dispersed (insoluble form) or molecularly dissolved (soluble form) throughout the Shell Composition 7 of the Capsule Cap 1 or Body 2. Capsule core is depicted in FIG. 2 as 8. The Capsule Core 8 is filled with Capsule Core Material 9. The Capsule core material 9 may contain a drug or a combination of drugs along with excipients. The Outer Surface of Capsule Cap 6 can be coated with 10 and the Outer Surface of Body can be coated with 11. In some embodiments, the coating materials 10 and 11 may be the same and in some cases, the coatings 10 and 11 may be different. In some cases, there may not be any coatings 10 or 11. At the region where the Capsule Cap 1 fits on the Capsule body 2, in some embodiments, a Band 12 is placed to seal the gap.

The present disclosure proposes a new way of combining more than one drug component in the capsule formulation. It may serve two purposes—prevent/reduce drug-drug interaction during storage of the dosage form and the release rate of each drug may be controlled to a desired value. The drug-loaded empty hard capsule shells, in which the active ingredient is physically dispersed or molecularly dissolved or chemically bound in the capsule shell, are then used further to manufacture pharmaceutical capsule formulations by filling them with drug-loaded powder, granules, beads, etc., i.e., the capsule core materials. In one embodiment, the drug is dissolved in the capsule shell-forming composition. In another embodiment, the drug which is insoluble in the Capsule Shell-forming composition is physically dispersed or suspended. In another embodiment, the drug may be partially dissolved and partially suspended in the capsule shell-forming composition. For the purpose of this disclosure, partially is defined as more than 10% of the total drug amount. For example, 30% drug may be physically dispersed, and 70% drug may be in the molecularly dissolved form. In yet another embodiment, the drug is chemically bound to the gelatin or polymer used in the Capsule shell-forming composition. In yet another embodiment, the drug is chemically bound to the gelatin or polymer used in the capsule shell-forming composition and also dissolved or suspended. The capsules are prepared by dip-coating or heat-melting methods.

The polymers that can be used in making the present hard capsule shells can be divided into the following groups: 1) Cellulose- or cellulose derived-based material, which include, but are not limited to, cellulose, cellulose ether, methyl cellulose, hydroxypropyl methyl cellulose, hydroxypropyl cellulose, hydroxyethyl cellulose, carboxymethyl cellulose, cellulose acetate phthalate. 2) starch-based compounds, which include, but not limited to hydroxyethyl starch, hydroxypropyl starch, hydroxyethyl methyl starch, 3) carrageenans—kappa and iota, 4) Acrylate derivatives which include, but not limited to, polyacrylate, polymethylacrylate, poly(acrylate-methylacrylate), poly(methyl acrylate-methyl methacrylate), 5) polyolefins which include, but not limited to, polyvinyl chloride, polyvinyl alcohol, and polyvinyl acetate and 6) pullulan (a polysaccharide polymer consisting of maltotriose units).

A hard capsule dosage form is manufactured by filling the core of the hard capsule shell with powders, granules, beads, pellets, a tablet, another capsule or combination thereof. In the recent times, scientists have started to fill the hard capsule shells with liquids too. The hard capsule shells are purchased from the capsule suppliers. There are main three manufacturers of hard capsule shells—Capsugel, Shionogi and Universal Capsules. Each capsule has two parts—a body and a cap. Commonly used primary ingredients in the composition of the capsule body or the cap are gelatin and hydroxypropyl methyl cellulose. As mentioned in the earlier section, apart from gelatin, several new types of polymeric substances have been used to manufacture hard capsule shells. These are—cellulosic compounds, acrylates, starch ethers, polyolefins, pullulans, and carrageenans. Apart from the main constituent of the capsule shell, gelatin or polymer, the shell may also contain other excipients such as plasticizers (e.g., polyethylene glycol, sorbitol, glycerol), stabilizers (antimicrobial and antioxidants), colorants (FD&C colors, titanium dioxide, natural dyes including riboflavin, carotenes, turmeric and caramel) and sequestering agents (citric acid, sodium citrate, ethylenediaminetetraacetic acid).

The rate of capsule dissolution in a desired media governs the release of medicaments residing in the core of the capsule. The rate of release may be altered using selected combination of polymers in the capsule composition or by a cross-linking of gelatin or polymer used in the capsule shell formulation.

The present disclosure proposes a method to develop a combination dosage form in which the medicament resides in the core of the capsule and another in the hard-capsule shell composition itself. The medicament in the hard-capsule shell is either physically dispersed or molecularly dissolved or chemically bound to the gelatin or to the polymeric material of the capsule shell. In one embodiment, the body and the cap of the capsule shell contain the same active moiety. In another embodiment, either the cap or the body of the capsule shell contains the medicament. In yet another embodiment, different medicaments are incorporated in the body and the cap. In yet another embodiment, a combination of medicaments is incorporated in the body and cap of the hard-capsule shell compositions.

According to the present disclosure, the hard capsule shell manufacturer may manufacture the capsule shell compositions containing a drug(s) and supply to a pharmaceutical company. The pharmaceutical company, purchasing the drug-loaded hard capsule shell compositions, will produce a suitable composition (such as powders, granules etc.) of the core material containing different drug(s) and fill it in the drug-loaded capsule shells.

There are various sizes of hard capsule shells available ranging from 000 to 5, the most commonly used are sizes 0 and 1. The fill weight of granules with a density of 0.7 g/mL is 475 mg and 350 mg for size 0 and 1 capsules, respectively. Size 000 capsule can contain 960 mg of core material loaded with medicament. The average weights of size 1 and 0 empty hard gelatin capsule shell are 75 mg and 98 mg, respectively. These weights can vary, but overall the weights are low. The drug loading in the capsule shell composition can affect the properties of the capsule shell; and thus, the amount of drug loaded in the capsule shell is limited. In general, only potent drugs can be loaded in capsule shells.

Also, one skilled in the art will determine the stability of the drug in the capsule shell composition during manufacture and storage. It is important to establish the desired release rate of medicament from the capsule shell under pre-determined conditions such as in the acidic and basic media, and in the presence of bile acids/food etc. The polymer for the capsule shell and the drugs need to be selected judiciously.

Bioavailability of a drug constitutes of two features—the rate and the extent of absorption. For drugs with narrow therapeutic indices, it is critical to maintain appropriate drug levels in the blood or tissues. For potent drugs, one must avoid dumping of drugs in a short period from the delivery device into the gastro-intestinal tract so that one can avoid erratic blood levels of the medicament.

The medicament, if insoluble, forms a suspension in the capsule shell-forming composition. The medicament may dissolve in the capsule shell-forming composition and in some cases, the medicament may form an ion-pair bond with the groups in the polymer or the excipients used to prepare the capsule shell-forming composition. The medicament may form a covalent bond with the polymer or the excipients used to prepare the capsule composition. The chemical bond can be of any nature—peptide, an amide, an ester or other kinds.

Overall, the drug may be present in the capsule-shell formulation in three states—physically dispersed, molecularly dissolved and chemically bound or combination of these.

In one embodiment, the drug may be in partially physically dispersed and molecularly dissolved states in the capsule-shell composition. In another embodiment, the drug may be in partially physically dispersed and chemically bound states in the capsule shell composition. In yet another composition, the drug may be in partially molecularly dissolved and chemically bound states in the capsule-shell composition.

Upon administration of the hard shell capsule orally, it may disintegrate or dissolve in the mouth cavity releasing the drug (s). In another embodiment, the capsule shell may stay intact in the mouth cavity and may dissolve or disintegrate in the stomach releasing the drug(s). In another embodiment, the capsule shell may stay intact till it reaches the intestinal tract and may dissolve/disintegrate in the intestine releasing the drug(s).

The release of drug, which is embedded in the capsule shell composition, may be controlled by three mechanisms—diffusion through the shell matrix, hydrolysis of medicament-polymer chemical bond or by dissolution of the hard-capsule shell to release the drug. In an embodiment, multiple drugs can be incorporated in the capsule shell composition, which could be released with different mechanisms. In another embodiment, a laminated hard shell capsule is prepared using the double pin-dip method. The drug-loaded polymer layer can be the inner or outer layer. One such type of lamination could be enteric coating. Enteric coating is a special coating that prevents release of the drug(s) from a dosage form until it reaches to the intestine.

Other medicaments may be placed in the capsule core in the form of powder, granules, beads, tablets, capsules etc.

The medicines used for oral contraception can be incorporated into the capsule shell matrix and/or carried inside the capsule based on the therapeutic amounts needed to treat the disease. Because the capsule shell properties might be negatively affected by the amount of drug incorporated in the capsule shell matrix, potent drugs are preferably (but not necessarily) chosen for incorporation into the capsule shell matrix. In this application, a “potent” drug is defined as drug with a therapeutic dose of 5 mg or less.

For potent drugs, the formulator face challenges to meet the content uniformity specifications. Content uniformity measures an even distribution of medicine in the drug delivery matrix. Because the therapeutic dose is very low for potent drugs, the drugs would be mostly in dissolved state in the capsule shell matrix solution.

The following oral contraceptives are available and used mostly in combination. For example, AVIANE-21® (ethinyl estradiol; levonorgestrel, 0.02 mg; 0.1 mg). The oral contraceptives such as Estradiol, Norethindrone acetate, Ethinyl Estradiol, Levonorgestrel, Drospirenone, Desogestrel, Norgestrel, Norgestimate, Dienogest, Norelgestromin, Ethynodiol diacetate are mostly given in the combination as it is more effective when given in combination. Many companies manufacture and market oral contraceptive products. Most of these are combination products. The doses of each product may vary from manufacturer to manufacturer.

Sometime, oral contraceptives are also give in combination with dietary supplements. Some examples of dietary supplements are ferrous fumarate, Levomefolate calcium etc. Mostly ferrous fumarate is given with Ethinyl estradiol and Norethindrone acetate. Many oral contraceptives may deplete several vitamins such as Riboflavin, B6, B12, folic acid, Vitamin C and minerals such as Magnesium and zinc. One can fill multivitamin mixture or a mixture of multivitamin and minerals in the capsule shells containing oral contraceptives. For the purpose of this patent, vitamins include vitamin A, D, E, K, B₁, B₂, B₃, B₅, B₆, B₇, B₉, B₁₂, C and minerals include calcium, phosphorus, potassium, sodium, sulfur, magnesium, iron, chlorine, cobalt, copper, zinc, manganese, molybdenum, iodine, selenium.

Table 2 lists the representative weights of different sizes of HPMC capsules, including weights of caps and bodies. It also lists the % weight of body compared to the total capsule weight.

TABLE 2
Representative weights of different sizes of HPMC capsules

| | Weight of | Weight of body, | Total capsule |
|---|---|---|---|
| Capsule size | Cap, mg | mg (% body weight) | weight, mg |
| 00 | 47.0 | 78.6 (62.5) | 125.6 |
| 0 | 39.1 | 57.5 (59.5) | 96.6 |
| 1 | 29.1 | 51.2 (63.8) | 80.3 |
| 2 | 25.6 | 36.9 (59.0) | 62.5 |
| 3 | 20.0 | 29.2 (59.3) | 49.2 |
| 4 | 14.5 | 22.8 (61.1) | 37.3 |
| 5 | 11.1 | 14.3 (56.3) | 25.4 |

It is clear that higher the capsule size, lower the capsule weight. Cap of the capsule weighs less than the body. The body weight is about 60% of the total capsule weight.

The concept of delivering the oral contraceptives distributed in the capsule shell matrix in this patent can be more clear or evident with the following non-limiting examples:

### Example 1: Estradiol in the Capsule Shell Matrix

The doses of Estradiol are only 0.5 and 1 mg per tablet. The drug, in these amounts, is soluble in the capsule shell matrix solution. The drug is also known to be heat stable. We can incorporate any of these strengths (0.5 and 1 mg) in the capsule shell composition forming any of the capsule sizes listed in Table 2. The drug can be incorporated throughout the capsule shell matrix (that is, be present in either the capsule cap and the capsule body), only in the capsule cap, or only in the capsule body. If the capsule is intended to deliver only Estradiol, the core of the capsule may be left empty. In another embodiment, the capsule is filled with a mixture of multivitamin and minerals.

### Example 2: Levonorgestrel in the Capsule Shell Matrix

The doses of levonorgestrel are only 0.1 to 1.5 mg per tablet. We can incorporate all these strengths in any of the capsule sizes listed in Table 2. The drug can be incorporated throughout the capsule shell matrix, only in the capsule cap of sizes 3 to 00 or only in the capsule body of sizes 4 to 00. If the capsule is intended to deliver only levonorgestrel, the core of the capsule may be left empty. In another embodiment, the capsule is filled with a mixture of multivitamin and minerals.

### Example 3: Norethindrone in the Capsule Shell Matrix

The doses of norethindrone are 0.01 mg to 0.35 mg per tablet. We can incorporate all these strengths in any of the capsule sizes listed in Table 2. The drug can be incorporated throughout the capsule shell matrix, only in the capsule cap or only in the capsule body. If the capsule is intended to deliver only norethindrone, the core of the capsule may be left empty. In another embodiment, the capsule is filled with a mixture of multivitamin and minerals.

### Example 4: Combination of Ethinyl Estradiol and Levonorgestrel

In this case, the doses of ethinyl estradiol and levonorgestrel 0.02 and 0.1 mg per tablet, respectively. In this example, estradiol is incorporated in the capsule shell matrix and levonorgestrel is filled into the capsule core. In this case, we use only capsule size 4 or 5. In another embodiment, ethinyl estradiol is incorporated in the capsule shell cap matrix and levonorgestrel is incorporated in the capsule shell body matrix.

### Example 5: Combination of Desogestrel and Ethinyl Estradiol

Ethinyl estradiol is incorporated in the capsule shell matrix and Desogestrel formulation is filled in the capsule shell core. As in Example 4, only sizes 4 or 5 capsules can be used for these lower doses.

### Example 6: Combination of Ethinyl Estradiol and Norethindrone Acetate

Ethinyl estradiol and Norethindrone acetate are incorporated in the capsule shell matrix as their respective doses are 0.03 and 1.5 mg, respectively. In this case, one can be filled in cap and the other can be filled in body. For this purpose, capsule sizes 3 or 4 or 5 can be used. In another embodiment, both drugs are distributed in the capsule shell matrix of cap and body.

### Example 7: Combination of Norgestrel, Ethinyl Estradiol and Ferrous Fumarate

The doses of norgestrel and ethinyl estradiol are 0.3 mg and 0.03 mg respectively. The amount of ferrous fumarate is 75 mg. Here, ferrous fumarate is used as inactive ingredient or placebo. So, out of 28 capsules, 21 capsules contain active as norgestrel and ethinyl estradiol while remaining 7 (which are placebo) contain only ferrous fumarate. It must be filled into the capsule core. Norgestrel and Ethinyl estradiol are molecularly dissolved in the capsule shell matrix (cap, body or both cap and body).

### Example 8: Combination of Drospirenone, Ethinyl Estradiol and Levomefolate Calcium

Levomefolate calcium is metabolite of vitamin B9 and prevent low folate levels. Drospirenone and ethinyl estradiol can be molecularly incorporated in the capsule shell matrix (cap or body or cap+body) and levomefolate calcium can be filled in capsule core.

### Example 9: Combination of Ethinyl Estradiol and Norethindrone to Produce Multiphasic Products

This example in Table 3 demonstrates how capsules can be prepared for monophasic, biphasic and triphasic product line. The capsules can be color coded to avoid confusion.

TABLE 3
Different formulations of ethinyl estradiol and norethindrone

| | | Color of | Color of |
|---|---|---|---|
| Phase | Dose, once a day capsule | capsule cap | capsule body |
| Monophasic | Ethinyl estradiol - 35 μg | Light Blue | — |
| | Norethindrone - 1.0 mg | — | Dark blue |
| Biphasic | Ethinyl estradiol - 35 μg | Light Blue | — |
| | Norethindrone - 0.5 mg | — | Red |
| | Ethinyl estradiol - 35 μg | Light Blue | — |
| | Norethindrone - 1.0 mg | — | Dark blue |
| Triphasic | Ethinyl estradiol - 35 μg | Light Blue | — |
| | Norethindrone - 0.5 mg | — | Red |
| | Ethinyl estradiol - 35 μg | Light Blue | — |
| | Norethindrone - 0.75 mg | — | Yellow |
| | Ethinyl estradiol - 35 μg | Light Blue | — |
| | Norethindrone - 1.0 mg | — | Dark blue |

Caps of capsules with light blue color are prepared and are used in all the formulations. Norethindrone is included in the body of the capsules in strengths 0.5 mg, 0.75 and 1.0 mg and their respective colors are red, yellow and dark Blue. A set of Caps and body of the capsules are prepared without any drug incorporated. Thus, when the cap without a drug is matched with the body containing three different strengths of norethindrone, we can generate three different strengths of norethindrone capsules. When the body without any drug is matched with the cap containing 35 μg of ethinyl estradiol, we get a capsule only with ethinyl estradiol.

From the foregoing, it is clear that this disclosure opens up several possibilities using the drug-loaded hard capsule shell as a carrier of different medicaments along with a different set of drugs in the core.

While specific embodiments have been presented here, various modifications can be made and the disclosure is not limited to the examples shown herein. Oral contraceptives may be incorporated in one of capsule body or cap or medicines or may be incorporated in each of the capsule body and cap. One may or may not fill the core with another drug, dietary supplement, complexing agent or other excipients. An empty drug-loaded capsule shell can also be used as a drug delivery system.

While one or more embodiments have been disclosed and described in detail, it is understood that this is capable of modification and that the scope of the disclosure is not limited to the precise details set forth but includes modifications obvious to a person of ordinary skill in possession of this disclosure, including (but not limited to) changes in material selection, drug selection and dosages, presence of the oral contraceptive drug(s) in the capsule composition or within the core defined by the capsule, size, operating ranges (temperature, volume, displacement, composition ingredients and ratios, and the like), environment of use, and also such changes and alterations as fall within the purview of the following claims.

1-11. (canceled)

12. A hard shell capsule for oral contraceptive in human beings comprising:
a body and a cap cooperatively defining a hollow core hard shell capsule; each of the body and the cap comprising a composition including a olymeric system and a drug, wherein the body further comprising a therapeutically effective amount of drug A loaded throughout the composition; the cap further comprising a composition comprising a therapeutically effective amount of drug B loaded throughout the composition; the body and cap compositions together containing a therapeutically effective amount of the drugs A and B; said drugs being oral contraceptive agents, the polymeric system of said body and cap compositions forming the hard polymer structure; wherein the core of the capsule is filled with therapeutically effective amount of a second drug(s).

13. The hard shell capsule of claim 12 wherein said drugs A and B are selected from the group of oral contraceptives, but are not limited to, Cyproterone acetate, Estradiol, Oestradiol, Norethindrone acetate, Ethinyl Estradiol, Levonorgestrel, Dienogest, Drospirenone, Desogestrel, Ethynodiol, Diacetate, Mestranol, Nomegestrol acetate, Norgestrel, Norgestimate, Dienogest, Norelgestromin, Norethisterone, Gestodene, Oestradiol valerate, and Ethynodiol diacetate.

14. The hard shell capsule of claim 12 wherein the core of the capsule holds (a) an additional therapeutic amount of a second drug(s); optionally (b) a dietary supplement; optionally (c) a complexing agent; and optionally (d) excipients.

15. (canceled)

16. (canceled)

17. (canceled)

18. (canceled)

19. (canceled)

20. (canceled)

21. (canceled)

22. (canceled)

23. The hard shell capsules of claim 12 wherein the colors of the cap and body are different.

24. The hard shell capsule of claim 14 wherein the dietary supplement is ferrous fumarate, levomefolate calcium, multivitamins, minerals and mixtures thereof.
